(19) United States
(12) Patent Application Publication
(10) Pub. No.: US 2018/0368676 A1
(43) Pub. Date: Dec. 27, 2018

(54) FUNDUS CAMERA

(71) Applicant: EasyScan B.V., 's-Gravenhage (NL)

(72) Inventors: Dirk Lucas De Brouwere, 's-Gravenhage (NL); Thomas Van Elzakker, 's-Gravenhage (NL)

(73) Assignee: EasyScan B.V., 's-Gravenhage (NL)

(21) Appl. No.: 16/064,026

(22) PCT Filed: Dec. 20, 2016
(86) PCT No.: PCT/NL2016/050895

§ 371 Date: Jun. 20, 2018

(30) Foreign Application Priority Data
Dec. 24, 2015 (NL) 2016037

(51) Int. Cl. A61B 3/00; G06T 7/70; G06F 3/16; A61B 3/12

(52) U.S. Cl. CPC A61B 3/0025; A61B 3/0091; A61B 3/12; A61B 3/0008; G06F 3/167; G06T 7/70

(57) ABSTRACT

A fundus camera includes a projector, an imaging unit, a processing unit and a feedback device. The projector is configured to project an image on the fundus of a patient, after which a reflected image from the fundus is acquired by the imaging unit, where the reflected image includes optical information from at least a part of the projected image and an imaged part of the fundus. The processing unit is configured to analyse the reflected image and to compare the reflected image with the projected image in order to obtain an image of the imaged part of the fundus. The feedback device is configured to provide information to a user, based on the analysed reflected image. The processing unit is configured to merge the reflected image and the second reflected image, in order to obtain a wide-field composition of the fundus.

### CROSS-REFERENCE TO RELATED APPLICATIONS

This application is the National Stage of International Application No. PCT/NL2016/050895, filed Dec. 20, 2016, which claims the benefit of Netherlands Application No. NL 2016037, filed Dec. 24, 2015, the contents of which is incorporated by reference herein.

### FIELD OF THE INVENTION

The invention relates to a fundus camera for obtaining an image of a fundus. The invention further relates to a method for obtaining an image of the fundus with the use of the fundus camera.

### BACKGROUND OF THE INVENTION

Such fundus cameras have become popular lately, since they provide a method by which relatively easy scanning of the fundus can be performed, just as with a known ophthalmoscope. However, fundus cameras also allow for storage of the images that were taken during the scanning. This allows for post-scanning evaluation of the fundus image.

The image of the fundus provides information about the physical state of the eyes of the patient. For example, a blurred fundus surface, rather than a sharp image with the retinal blood vessels clearly shown, indicates that the patient might have glaucoma.

Fundus cameras are known, for example from WO2004/041120. This document discloses a method for acquiring images of the ocular fundus. With the method, it can be determined which areas of the fundus have been imaged and which areas still have to be imaged in order to obtain a wide-field composition of the fundus. The software disclosed in WO2004/041120 is configured to provide feedback on the basis of the captured images and to make the patient shift their line of sight in order to illuminate other parts of the fundus.

In an embodiment, the feedback provided to the patient comprises audible instructions on where to shift the line of sight. In another embodiment, the feedback can comprise a moveable illumination source, whereby the patients need to follow the moveable source in order to change their line of sight. When the line of sight of the patient is shifted, the fundus is shifted with respect to the imaged area and different parts of the fundus become illuminated. The disclosed method is configured to stitch together the images from the different illuminated sights. This poses the advantage that a wide-view image of the fundus can be reconstructed with images from a narrow-view, but higher quality, camera, due to multiple stitched images.

With such a method, an operator is required to operate the instrument, for example for the focussing of the sight of the patient. It is in fact important to have a good focus on the fundus surface, since otherwise no clear image be taken that exposes the required details on the fundus surface. For the patient, it can be difficult to focus his eyes by himself, since the projected illumination source only is a light source, which poses little contrast to aid the focussing.

There is a continuous need for providing improved fundus cameras of more simple construction or having more accurate or reliable results in particular there is a continuous need for fundus cameras that can be easily operated by the user.

### SUMMARY OF THE INVENTION

The present invention provides a fundus camera. The invention further relates to method for obtaining an image of the fundus.

The fundus camera is configured to obtain an image of a fundus of an eye of a patient. The fundus camera comprises a projector, which is configured to project an image on the fundus. The projector comprises a light source, which is configured to illuminate the fundus. In an embodiment, the projector may further comprise an optical filter and/or one or more lenses in order to obtain a desired image that can be projected on the fundus.

The fundus camera comprises an imaging unit, which is configured to acquire a reflected image. The imaging unit can, for example, be a digital optical sensor that is configured to transfer an incoming optical image into a digital output. The reflected image comprises at least a part of the projected image after reflection on at least a part of the fundus.

This part of the fundus is the part on which the projected image is projected and which reflects a portion back as the reflected image into the imaging unit. Since the projected image is in general wider than the sensor of the imaging unit, not all light from the projected image can be acquired by the imaging unit. Therefore a portion of the projected image is scattered after reflection on the fundus rather than being acquired by the imaging unit.

The fundus is known, from prior art, to be a reflective surface for light in the visible regime. The reflected image further comprises optical information of the fundus itself, since the fundus became illuminated and reflected the projected image accordingly.

The fundus camera comprises a processing unit, which is connected to the imaging unit. In an embodiment, this connection is an electrical connection, since the transferred signal from the imaging unit is a digital, electric signal. The processing unit is configured to analyse the reflected image. With this analysis, the structure of the fundus can be extracted from the reflected image, since fundi generally have a distinct structure, in particular due to blood vessels that are present below the fundus surface, which are visible in images of the fundus.

The fundus camera comprises a feedback device, which is configured to provide instructions to the user. These instructions are based on the analysed reflected image and can, for example, comprise information and instructions with which the user is suggested to change the position of the eye, such that the quality of the reflected image of the fundus may be improved.

The processing device and/or the feedback device may be part of the imaging unit.

In an embodiment, the feedback device is configured to provide optical instructions, wherein the projector is configured to provide the optical instructions of the feedback device.

The processing unit is configured to compare the reflected image with the projected image in order to obtain an image of at least a part of the fundus. Thereto, the projector is connected to the processing unit and the projected image is transmitted to the processing unit. The reflected image comprises optical information about both the projected image and the surface of the fundus. When the reflected image is compared with the projected image, the optical information of the projected image can be removed out of the reflected image, after which only an image of the imaged part of the fundus remains.

The comparison in the processing unit between the projected image and the reflected image may comprise an unwrapping algorithm, wherein the reflected image is deconvoluted with the projected image. In the embodiment wherein the sensor element of the imaging unit is smaller than the projected image, the reflected image is generally smaller than the projected image. In such case, the processing unit is configured to correct the projected image for this difference in image size.

In an embodiment, the instructions to the user comprise optical instructions. The optical instructions are presented to the fundus of the eye of the user, since the projected image comprises the optical instructions. As a result, the instructions can be observed by the user during the scanning of the fundus and the user can follow the instructions in-situ, during the scanning of the fundus. Such instructions can be given by the fundus camera autonomously, rather than by an operator.

In an embodiment, the optical instructions comprise an arrow which can be directed across the projected image. The arrow provides information to the user on where to align his line of sight. The direction and position of the arrow can be changed over time, such that images are obtained from many individual parts of the fundus. The optical instructions may further comprise an instruction presentation on how to use the fundus camera. The instructions can be configured to present possible results of the scanned fundus and present a diagnosis to the user.

In an embodiment, the optical instruction is a tracking target that is configured to move across the projected image and is intended to be aligned with the patient's line of sight. Such a tracking target can for example be a point that has a different colour as compared to the background. The object of the tracking target is that the user will follow it across the projected image and thereby changes its line of sight with respect to the camera, and in particular changes its line of sight with respect to the imaging unit.

In an embodiment, the instruction is intended to instruct a patient directly or indirectly to adapt a line of sight of the eye with respect to the camera in order to acquire a second reflected image of a second part of the fundus. When the instruction is followed-up by the user, in particular when the tracking target is tracked by the user across the projected image, the line of sight of the user is changed and the fundus is moved, e.g. rotated, with respect to the camera. Therefore, the acquired reflected image is reflected from a second part of the fundus, which is different from the first part of the fundus. The second image therefore comprises different optical information about the fundus since the image is acquired from a different parts of the fundus.

In an embodiment, the imaged part of the fundus and the second imaged part of the fundus at least partially overlap each other. Therefore, the reflected image and the second reflected image overlap, which has the advantage that the relative position between the images can be determined. In order to reach the overlap between the imaged part and the second imaged part of the fundus, the feedback device is configured to provide an optical instruction which is intended to adapt the line of sight of the user slightly.

In an embodiment, the processing unit is configured to merge the reflected image and the second reflected image in order to obtain a wide-field composition of the fundus. The overlap between the reflected image and the second reflected image allows that the images can be merged or stitched together. When the two images are merged, a larger image is created in which the part of the fundus and the second part of the fundus are displayed together.

An advantage of the merging of several smaller reflected images in a wide-field composition is that the sensor part of the imaging unit can be made smaller as compared to when entire fundus had to be imaged. In another case, the optics that transmit the reflected images, need to converge less, which allows for higher quality optics and, resulting from that, higher quality images.

In an embodiment, the line of sight can be adapted multiple times, so as to obtain a third reflected image, a fourth reflected image and so on. The more reflected images are acquired, the larger the total imaged part of the fundus will become.

In an embodiment, the projector is configured to project multiple images, each having a different illumination pattern which can be used for a different type of functional diagnosis of the fundus. The different illumination patterns can be used for the diagnosing of multiple aberrations in the fundus. For example, a map with topographic data of the fundus can be obtained when a structured pattern of parallel lines is projected on the fundus. A pattern with a changed structure, with for example a different spacing between the parallel lines, is then shown in the reflected image, because the surface of the fundus is not flat. The nature of the pattern with the changed structure and the spacing in between lines is then a measure for the structure of the fundus. Functional analysis of photoreceptors in the fundus can be performed when the fundus is flashed with a checkerboard pattern, since a different reflectivity of the fundus will occur before, during and after the flashing.

In an embodiment, the projected image is a video, wherein the projector is a video projector, which is configured to project the video. The video comprises a multitude of different projected pictures per unit time, wherein it appears to the user that a smooth transition between images occurs. As a result, the optical instructions comprise for example a moving target that is shifted through the projected image in such a way, that the user would observe a continuous movement rather than a discrete movement.

In an embodiment, the camera is configured to be operated by the patient and a separate operator is no longer required. The operation of the camera therefore needs to be simple and clear. Thereto, the camera may comprise a single user interface with which all the required information for the imaging of the fundus, such as personal information and known eye aberrations, can be fed into the camera and on which the obtained image of the fundus can be shown to the patient.

An advantage of operation by the patient is that the fundus camera can be placed in public areas, rather than in specified locations where operators are available. The camera is thereby configured to provide a self-diagnose for the patients, whenever they want.

In an embodiment, the instructions to the user comprise information on the desired location of the focussing point of the line of sight of the eye. In order to obtain a sharp image of the fundus, it is required that the eye is in focus with the imaging unit. Therefore, the instructions are intended to let the patient focus their line of sight in the axial direction.

Furthermore, the fundus camera is configured to obtain optical autofocus by the lateral displacement of an illumination pattern, which is caused by a parallax that is created by a difference between the projected image and the reflected image.

In an embodiment, the feedback device comprises an acoustic device, which is configured to provide instructions to the user with an audible instruction signal. The audible signal may be provided parallel to an optical instruction. The audible signal may be an audible text and can for example be used to instruct the user about the progress of the scanning and/or to instruct the patient to blink or not.

In an embodiment, the fundus camera is a hand-held device.

The invention further provides a method for obtaining an image of the fundus with the use of a fundus camera according to the present invention.

The method comprises the step of aligning of the line of sight of the eye of the patient with the projected image of the fundus camera. In this step, the patient will present its head in front of the fundus camera and the eye, that needs to be tested, in front of the projector and the imaging unit. During this step, the patient should visualize the projected image on his fundus.

The patient is requested to adapt his line of sight to the projected image, or in particular to a desired location in the projected image. The fundus camera will provide this desired location, which can be, in an embodiment, the centre of the projected image. Furthermore, the patient should focus its sight, such that he observes a sharp projected image, rather than a blurred image.

The method comprises the step of acquiring the reflected image of the projected image from the fundus. During this step, the reflected image is acquired by the imaging unit, in particular by a sensor part of the imaging unit.

The method comprises the step of comparing the reflected image and the projected image in order to obtain an image of the part of the fundus. This comparison is done by the processing unit, which is configured to obtain an image of the fundus by subtracting the optical information of the projected image from the reflected image, such that only optical information of the fundus is remained after this step.

Based on the acquired image of the part of the fundus, the processing unit is configured to determine what part of the fundus should be imaged next in order to obtain the required image of the fundus.

The method comprises the step of providing one or more instructions to the user. These instructions may be intended to change the line of sight of the patient, after which another reflected image can be obtained from a different part of the fundus.

The instructions to the user can, in another case, comprise the instruction to blink his eye or to remove his eye away from the camera, for example in case the imaging of the fundus is over. Such an instruction can, in an embodiment, be provided by means of an audible instruction signal, in particular an audible text.

In an embodiment, the method comprises the step of projecting one or more optical instructions in the projected image. The patient can thereby see the instructions with his test eye. Such optical instructions may comprise a tracking target which can be tracked by the patient in order to change his line of sight and to change the part of the fundus from which the projected image is reflected, forming the reflected image.

In an embodiment, the method comprises repeating the steps of acquiring, comparing, providing and projecting. This repeating allows the imaging unit to acquire multiple images of the same or different parts of the fundus. The multiple images may cover a larger area of the fundus as compared to the area of the fundus that is covered by a single image. In an embodiment, the imaged parts of the fundus, for each of the multiple images, overlap with at least one of the other imaged parts. The processing unit is configured to determine the relative position of each of the multiple images by using the overlap.

In an embodiment, the method comprises the step of merging the reflected image and the second reflected image in order to obtain a wide-field composition of the fundus. When multiple reflected images from different parts of the fundus are merged, the obtained wide-field composition comprises a detailed image of the fundus in which, in the meantime, a large portion of the fundus is displayed. The conclusion and/or diagnosis about the state of the fundus and possible aberrations can be determined by the processing unit, based on the obtained wide-field composition.

Further characteristics and advantages of the fundus camera according to the invention will be explained in more detail below with reference to an embodiment which is illustrated in the appended drawings.

### BRIEF DESCRIPTION OF THE DRAWINGS

FIG. 1 schematically depicts an embodiment of a fundus camera according to the invention;

FIG. 2 schematically depicts an embodiment of fundus camera, wherein the tracking target is arranged in the centre of the projected image;

FIG. 3 schematically depicts an embodiment of fundus camera, wherein the tracking target is arranged in a more left portion of the projected image; and

FIG. 4 schematically depicts an embodiment of fundus camera, wherein the tracking target is arranged in a more right portion of the projected image.

### DETAILED DESCRIPTION OF THE INVENTION

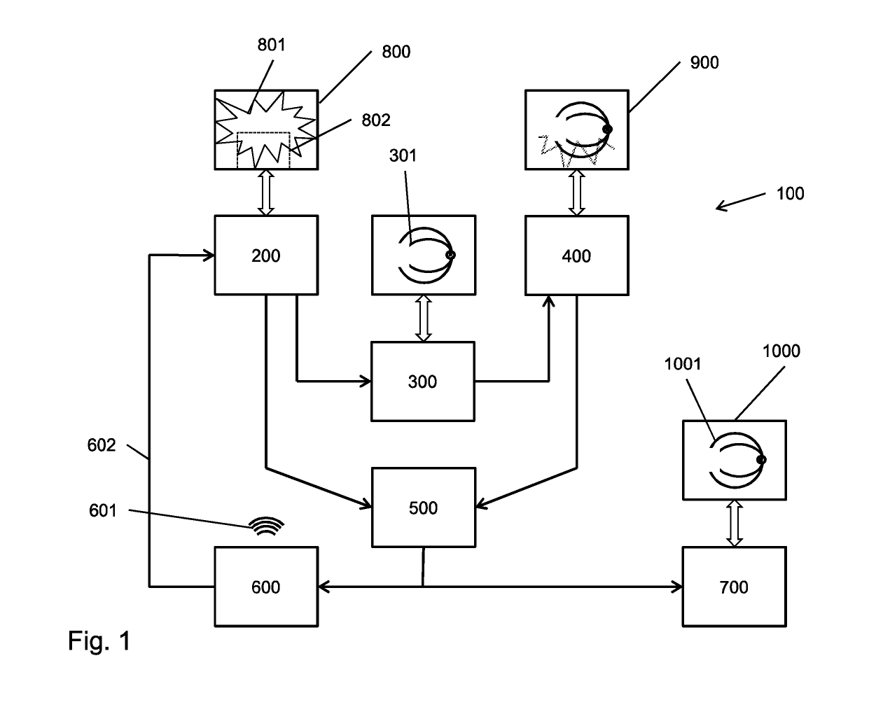

FIG. 1 discloses a schematic representation of a fundus camera according to the invention, denoted by reference numeral 100. The fundus camera 100 comprises a projector 200, which is configured to project an image 800 on a fundus 300 of an eye of a patient. In the shown embodiment, the projected image 800 comprises a projected pattern 801.

The fundus camera 100 comprises an imaging unit 400, which is configured to acquire a reflected image 900, wherein the reflected image 900 comprises at least a part of the projected image 800 after reflection on the fundus 300. Therefore, the reflected image 900 comprises optical information from the projected image 800, in particular a portion 802 of the projected pattern 801, and from the fundus 300.

The fundus camera 100 comprises a processing unit 500, which is connected to the imaging unit 400 and is configured to analyse the reflected image 900. The processing unit 500 is for example configured to extract the structure 301 of the fundus 300 from the reflected image 900, since the fundus 300 generally has a distinct structure 301. In particular, blood vessels are present below the fundus 300 surface, which are visible in images of the fundus 300.

In the embodiment, the processing unit 500 is connected to the projector 200, wherein this connection is configured to transmit the projected image 800 from the projector 200 to the processing unit 500. The processing unit 500 is configured to compare the reflected image 900 with the projected image 800 in order to obtain an image of at least a part of the fundus 300.

Since the reflected image 900 comprises optical information from the projected image 800 and the fundus 300, an image of the fundus 300 can be obtained when the optical information from the projected image 800 were to be removed from the reflected image 900.

Due to this comparison between the projected image 800 and the reflected image 900, the projected image 800 may comprise a projected pattern 801, rather than a white-light image, since the processing unit 500 is configured to subtract the image of the fundus 300 when the projected image 800, and in particular the projected pattern 801, is known.

The fundus camera 100 comprises a feedback device 600, which is configured to provide instructions to a user, wherein the instructions are based on the analysed reflected image 900.

In the embodiment, the instructions comprise an audible instruction signal 601, which is emitted by an acoustic device. The audible instruction signal 601 comprises information to instruct the user where to align his line of sight and information on the imaging process, such as whether the imaging has stopped and the user is allowed to move his eye away from the fundus camera 100.

In the shown embodiment, the instructions further comprise optical instructions 602, which are projected on the fundus 300 with the projector 200. In the embodiment, the optical instructions 602 are merged with the projected image 800, such that the projected pattern 801 comprises the optical instructions 602. The optical instructions 602 comprise information to the user on where to align his line of sight, such that part of the fundus 300, from which the reflected image 900 is obtained, is changed during the imaging process.

In the shown embodiment, the optical instructions 602 in the projected pattern 801 of the projected image 800 comprise a tracking target which is intended to be followed by the line of sight of the patient.

The fundus camera 100 is configured to acquire images of multiple parts of the fundus 300, wherein the processing unit 500 is configured to merge the different images into a larger wide-field composition of the fundus 300.

In the embodiment, the fundus camera 100 is configured to be operated by the patient. The camera 100 therefore comprises a user interface 800 with which all the required information for the imaging, such as age, personal details and readily known fundus 300 aberrations, can be fed into the camera 100. The user interface 700 is configured to display an image 1000 of the wide-field composition of the fundus 300, in which the structure 1001 of the fundus 300 can be seen.

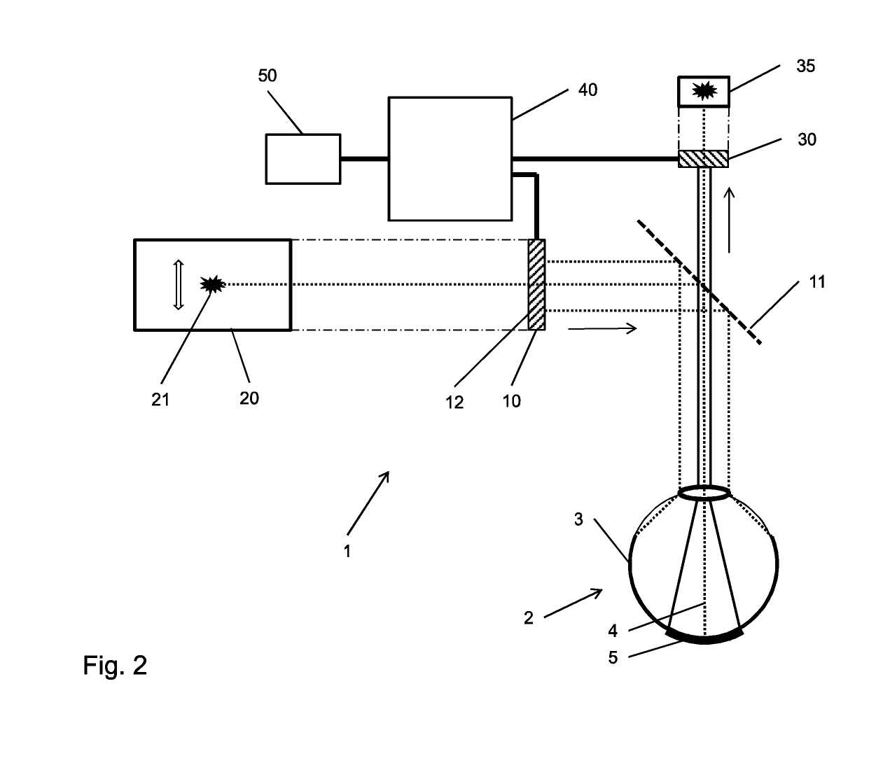

FIGS. 2, 3 and 4 disclose a schematic, top-view, representation of another embodiment of a fundus camera 1. The fundus camera 1 comprises a projector 10, which is configured to project an image 20, through a partially reflective mirror 11, on a fundus 3 of an eye 2 of a patient.

The embodiment of the fundus camera 1 comprises an imaging unit 30, which is configured to acquire a reflected image 35 from the fundus 3. The reflected image 35 comprises at least a part of the projected image 20 after reflection on at least a part 5 of the fundus 3.

The embodiment of the fundus camera 1 comprises a processing unit 40, which is connected to the imaging unit 30 and is configured to analyse the reflected image 35. The processing unit 40 is connected to the projector 10 as well, wherein this connection is configured to transmit the projected image 20 from the projector 10 to the processing unit 40. The processing unit 40 is configured to compare the reflected image 35 with the projected image 20 in order to obtain an image of the imaged part 5 of the fundus 3.

Since the reflected image 35 comprises optical information from the projected image 20 and the imaged part 5 of the fundus, the image of the imaged part 5 of the fundus 3 can be obtained when the optical information from the projected image 20 were to be subtracted from the reflected image 35.

The embodiment of the fundus camera 1 comprises a feedback device 12, which is arranged within the projector 10 and wherein the feedback device 12 is configured to provide an optical instruction to the user, based on the analysed reflected image from the processing unit 40. The optical instruction is a tracking target 21 that is configured to move across the projected image 20 and is intended to be aligned with the patient's line of sight 4.

The embodiment of the fundus camera 1 is configured to be operated by the user and comprises thereto a user interface 50 through which the fundus camera 1 can be operated. With the user interface 50, the required information for the imaging of the eye 2 can be fed into the camera 1. Furthermore, the user interface 50 is configured to display an image of the fundus 3.

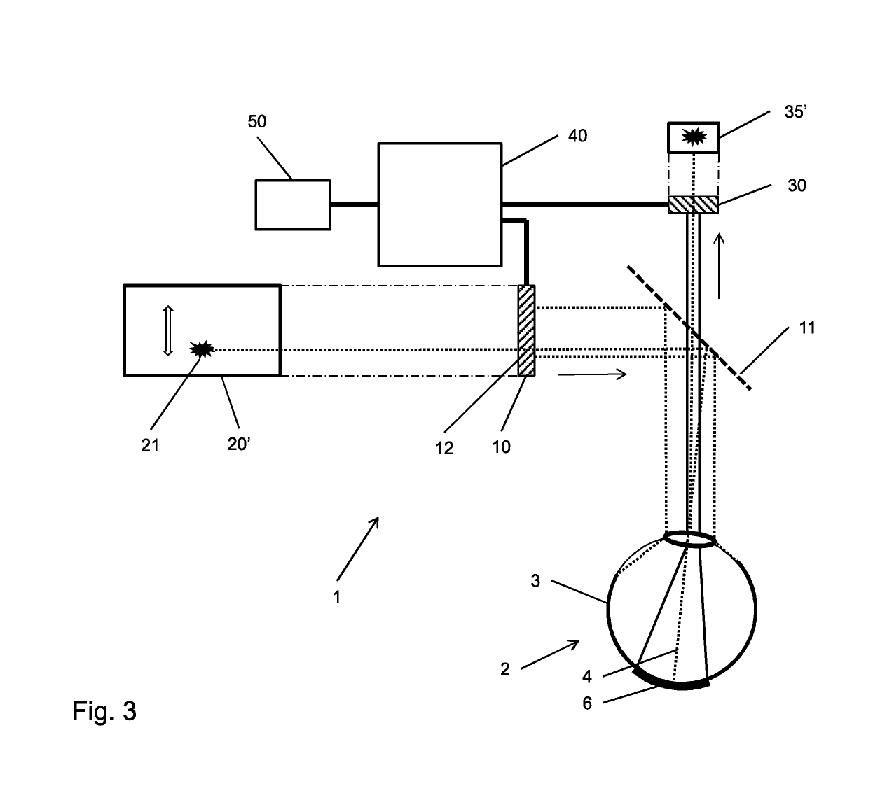

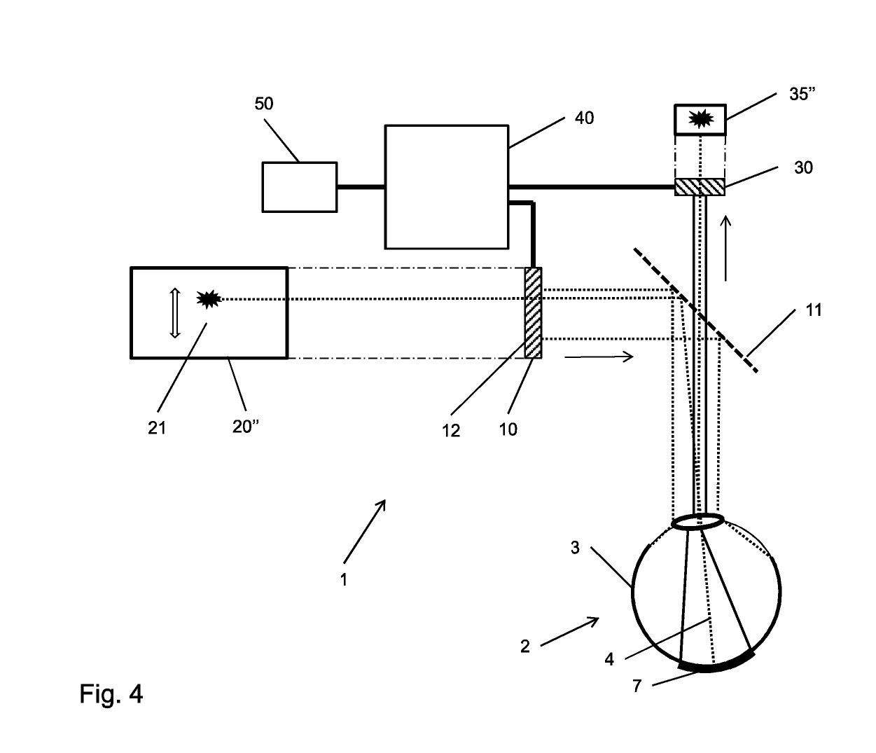

In FIG. 2, the tracking target 21 is displayed in the centre of the projected image 20. However, in FIGS. 3 and 4, the tracking target 21 is displayed in respectively a more left and a more right portion of the projected image 20. In order to track the tracking target 21 with the line of sight 4, the eye 2 of the patient is tilted in FIGS. 3 and 4 relative to the camera 1 as compared to the position of the eye 2 in FIG. 2.

Due to this change in the line of sight 4 of the eye 2, the reflected image 35′ is reflected from a different part of the fundus 2. When the tracking target 21 is moved to a left portion of the projected image 20′, as in FIG. 3, the eye 2 is tilted slightly to the right and the reflected image 35′ is obtained from a second part 6 of the fundus 3, which is arranged to the right of the imaged part 5 of the fundus 3. The second part 6 and the imaged part 5 of the fundus overlap each other at least partially.

When the tracking target 21 is moved to a right portion of the projected image 20″, as in FIG. 4, the eye 2 is tilted slightly to the left and the reflected image 35″ is obtained from a third part 7 of the fundus 3, which is arranged to the left of the imaged part 5 of the fundus 3. The third part 7 and the imaged part 5 of the fundus overlap each other at least partially.

The processing unit 40 is configured to merge the reflected image 35 from the imaged part 5 with the second reflected image 35′ from the second part 6 of the fundus 3 and the third reflected image 35″ from the third part 7 of the fundus 3 in order to obtain a wide-field composition of the fundus 3.

In the embodiment, the processing unit 40 is configured to determine which additional part of the fundus 3 needs to be imaged in order to obtain the desired wide-field composition of the fundus 3. Therefore, the processing unit 40 is configured to control the feedback device 12 such that the tracking target 21 is moved across the projected image 20 to a position wherein the line of sight 4 of the patient is directed such that the reflected image 35 is acquired from the desired part of the fundus 3.

It is remarked that in the above embodiments, the projector 200, the imaging unit 400, the processing unit 500, the feedback device 600 and the user interface 700 are shown as separate devices.

In practice, one or more of these devices may be integrated or housed in a single housing. In a preferred embodiment, all devices are housed in a single housing wherein two or more devise may be integrated as a single device.

1. A fundus camera for obtaining an image of a fundus, comprising:
a projector, which is configured to project an image on the fundus of an eye of a patient, wherein the projector comprises a light source;
an imaging unit, which is configured to acquire a reflected image comprising at least a part of the projected image after reflection on at least a part of the fundus;
a processing unit, connected to the imaging unit and configured to analyse the reflected image; and
a feedback device, which is configured to provide instructions to a user, based on the analysed reflected image,
wherein the processing unit is configured to compare the reflected image with the projected image in order to obtain an image of at least a part of the fundus.

2. The fundus camera according to claim 1, wherein the instructions to the user comprise an optical instruction, wherein the projected image comprises the optical instruction.

3. The fundus camera according to claim 2, wherein the optical instruction is a tracking target that is configured to move across the projected image and is configured to instruct the patient to align the patient's line of sight with the tracking target.

4. The fundus camera according to claim 1, wherein the instruction is intended to instruct the patient directly or indirectly to adapt a line of sight of the eye with respect to the camera in order to acquire a second reflected image of a second part of the fundus.

5. The fundus camera according to claim 4, wherein the imaged part of the fundus and the second imaged part of the fundus at least partially overlap each other.

6. The fundus camera according to claim 5, wherein the processing unit is configured to merge the reflected image and the second reflected image in order to obtain a wide-field composition of the fundus.

7. The fundus camera according to claim 1, wherein the projector is configured to project multiple images, each having a different illumination pattern which can be used for a different type of functional diagnosis of the fundus.

8. The fundus camera according to claim 1, wherein the projected image is a video.

9. The fundus camera according to claim 1, wherein the camera is configured to be operated by the patient.

10. The fundus camera according to claim 1, wherein the instructions to the user comprise information on the desired location of the focussing point of the line of sight of the eye.

11. The fundus camera according to claim 1, wherein the feedback device comprises an acoustic device, which is configured to provide instructions to the user with an audible instruction signal.

12. A method for obtaining an image of the fundus with the use of a fundus camera according to claim 1 comprising the steps of:
aligning of a line of sight of an eye of a patient with the projected image of the fundus camera;
acquiring the reflected image of the projected image from the fundus;
comparing the reflected image and the projected image in order to obtain an image of the part of the fundus; and
providing one or more instructions to the user.

13. The method according to claim 12, wherein the method comprises the step of projecting one or more instructions in the projected image.

14. The method according to claim 13, wherein the method comprises repeating the steps of acquiring, comparing, providing and projecting.

15. The method according to claim 14, wherein the method comprises the step of merging the reflected image and the second reflected image in order to obtain a wide-field composition of the fundus.
